(19) United States
(12) Patent Application Publication
(10) Pub. No.: US 2013/0023798 A1
(43) Pub. Date: Jan. 24, 2013

(54) METHOD FOR BODY-WORN SENSOR BASED PROSPECTIVE EVALUATION OF FALLS RISK IN COMMUNITY-DWELLING ELDERLY ADULTS

(71) Applicants: Barry R. GREENE, Dublin (IE); Emer P. Doheny, Dublin (IE)

(72) Inventors: Barry R. GREENE, Dublin (IE); Emer P. Doheny, Dublin (IE)

(73) Assignee: INTEL-GE CARE INNOVATIONS LLC, Roseville, CA (US)

(21) Appl. No.: 13/186,709

(22) Filed: Jul. 20, 2011

(51) Int. Cl. A61B 5/11

(52) U.S. Cl. 600/595

(57) ABSTRACT

Methods and systems may provide for falls risk assessment using body-worn sensors. If executed by the processor, the instructions can cause the system to calculate a timed up and go (TUG) time segment based on angular velocity data from the plurality of kinematic sensors. The instructions may also cause the system to calculate one or more derived parameters based on the angular velocity data, including temporal gait parameters, spatial gait parameters, tri-axial angular velocity parameters, and turn parameters. Falls data may be collected retrospectively, based on whether the test participant has fallen in the past. Falls data may be collected prospectively, in which the individual is contacted in the future to determine if they have fallen. This outcome data may be used to train regularized discriminant classifier models based on relevant sub-sets of the feature set, selected using sequential forward feature selection. Regularized discriminant parameters and along with associated sequential forward feature selection obtained feature set are obtained via grid-search

### BACKGROUND

1. Technical Field

Embodiments generally relate to prospectively assessing falls risk.

2. Discussion

Falls in the elderly may represent a substantial health care problem worldwide. Indeed, a significant percentage of people over seventy years of age experience a significant fall, and the frequency of falls increases with age and the level of frailty. The timed up and go (TUG) test was developed as a tool to screen for balance problems in older individuals. In the TUG test, the individual gets up from a chair, walks three meters, turns at a designated spot, returns to the seat and sits down, wherein the total time taken to perform the test may generally be considered as indicative of the frailty of the individual. While it may be generally inferred that elders with longer TUG times can be more likely to fall than those with shorter TUG times, there still remains considerable room for improvement with regard to the use of the TUG test to conduct falls risk assessments.

### BRIEF DESCRIPTION OF THE DRAWINGS

The various features of the embodiments will become apparent to one skilled in the art by reading the following specification and appended claims, and by referencing the following drawings, in which:

FIG. 1A illustrates a perspective view of an example of an individual performing the timed up and go (TUG) test;

FIG. 1B illustrates an embodiment of a gyroscope that can be used in acquiring parameters related to falls risk assessment.

FIG. 1C illustrates example recorded TUG times as a function of age and gender.

FIG. 2 illustrates a flowchart of an example of a method of generating TUG parameters from gyroscope data according to an embodiment;

FIG. 3 illustrates plots of examples of vertical (Z sensor axis), antero-posterior (X sensor axis), and medio-lateral (Y sensor axis) angular velocity according to an embodiment;

FIG. 4 illustrates plots of examples of left and right shank medio-lateral angular velocity according to an embodiment.

FIG. 5 illustrates example measured angular velocity signals and their use in determining turning properties.

FIG. 6 illustrates a model in measuring stride length.

FIG. 7 illustrates example parameters that may be included in a falls risk classifier model.

FIG. 8 illustrates the number of falls experienced by TUG test participants during a time span after the test.

FIG. 9 illustrates receiver operating characteristic (ROC) curves related to classifier models for falls risk assessment.

FIG. 10 illustrates a graphical representation of a grid search.

FIG. 11 illustrates ROC curves related to classifier models for falls risk assessment.

FIG. 12 illustrates a block diagram of an example of a computing system according to an embodiment.

### SUMMARY

Embodiments of this invention relate to generating models that assess a person's risk of falling and finding the optimum set of parameters and parameter values for the models.

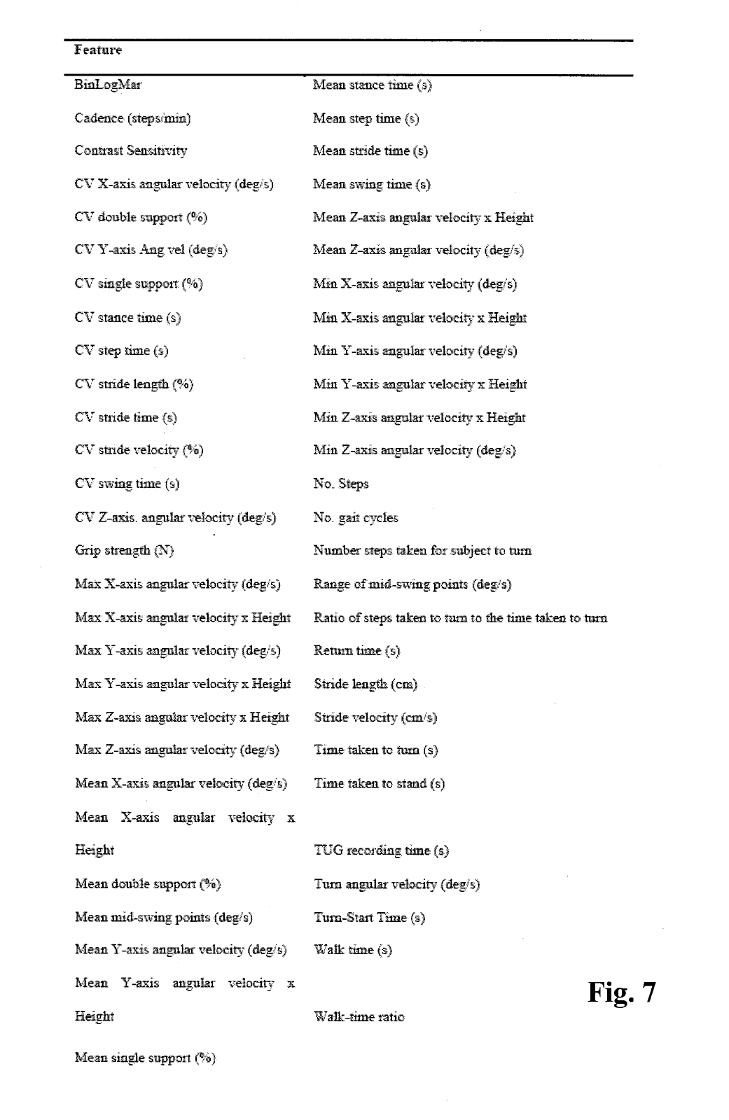

The parameters may be generated from sensors, such as gyroscopes and accelerometers, that are attached to a person's body, such as to one or both of the person's shanks, and that generate angular velocity data as the person moves. Some embodiments generate such data through conducting a timed up and go (TUG) test for a person. The parameters that may be generated and derived are discussed below in more detail and may be seen in FIG. 7. Examples include the number of steps taken by the person during the TUG test, the stride length, stride velocity, and time taken to complete the TUG test.

Classifier models may be generated to estimate risk of falling for all test participants, for a subset of test participants, or for a particular participant. The classifier models do not need to depend on all the derived parameters. Sequential forward feature selection may be used to identify an optimal set of parameters for the classifier models. A grid search may be used to identify optimal values for the parameters.

The classifier models may be trained using the actual occurrence of falls experienced by the test participants associated with the model. In a retrospective approach, the occurrence of falls may be based on a test participant's self reported falls history. Because such reports may be unreliable, however, a prospective approach may instead or in addition be used in which test participants are later asked if they experienced a fall. For example, after the original TUG test, test participants may be contacted and surveyed as to whether they experienced a fall and the number of falls they experienced. This data can be used to train the classifier models. The prospective approach may be used when more reliability in the training data is desired.

The resulting trained classifier models produce an estimate of falls risk, but their selected feature sets may also indicate particular parameters that affect the specific participants associated with the model. Because such parameters may relate to specific physical, sensory, or other deficits in a participant's movement, the parameters may allow a more targeted diagnosis and treatment to be applied to a participant seeking to lower the risk of falling.

### DETAILED DESCRIPTION

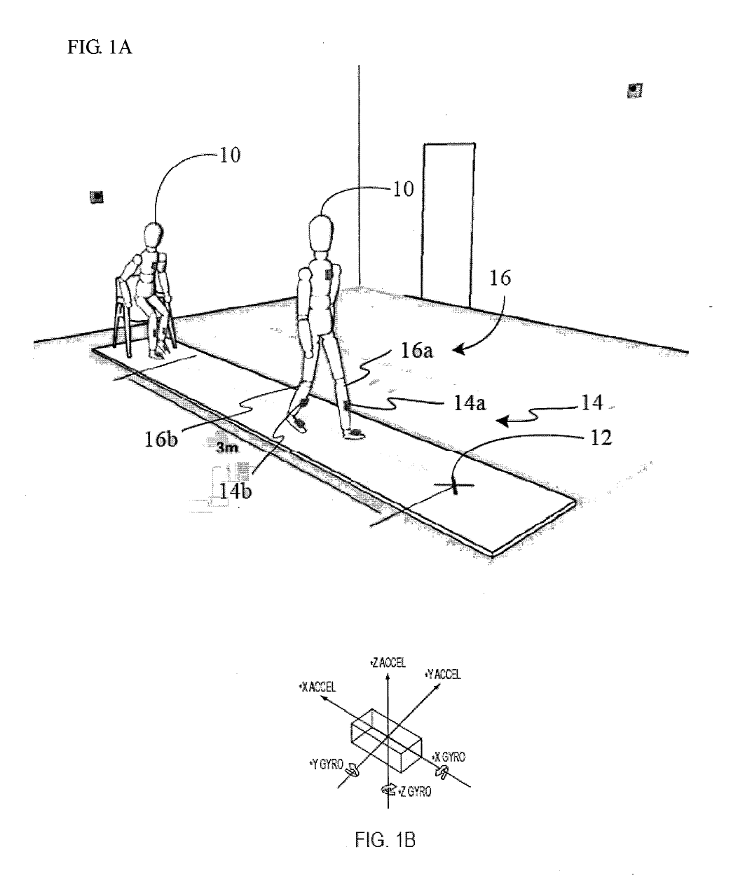

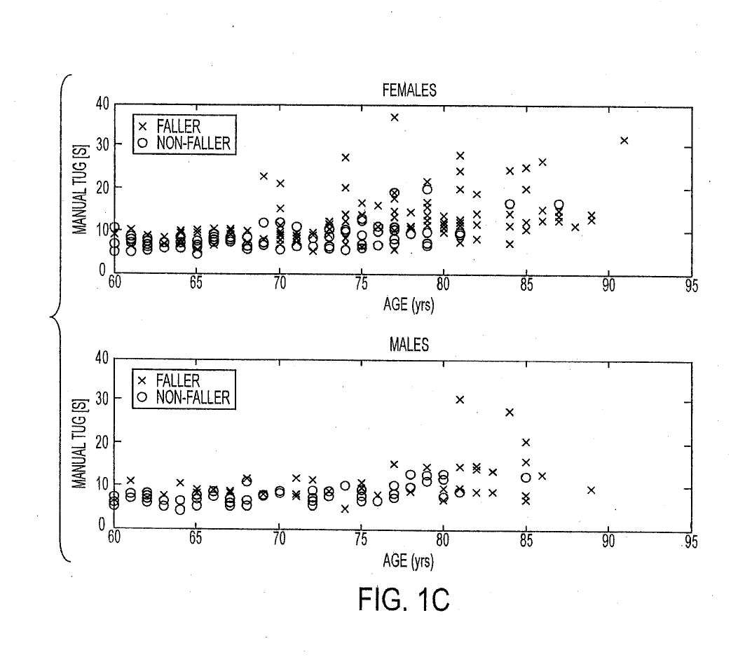

Embodiments may provide for a system including a plurality of inertial sensors to be coupled to a corresponding plurality of shanks of an individual, a processor, and memory to store a set of instructions. If executed by the processor, the instructions cause the system to calculate a timed up and go (TUG) time segment (discussed below in more detail) based on angular velocity data from the plurality of inertial sensors, and calculate a derived parameter based on the angular velocity data, which may be based on a measure of rotation about X, Y, and Z gyroscope sensor axes, as illustrated in FIG. 1B The TUG time segment may be generated based on recording the times in which participants get up from a chair, walk a distance (e.g., 3 meters), turn at a designated spot, return to the chair, and sit down. FIG. 1C illustrates example TUG times among male and female test participants. Further, the instructions, if executed, may also cause the system to generate a baseline falls risk assessment based on at least one of the TUG time segment and the derived parameter.

Embodiments may also provide for a computer readable storage medium including a set of instructions which, if executed by a processor, cause a computer to calculate a TUG time segment based on angular velocity vectors from the plurality of inertial sensors, and calculate a derived parameter based on the angular velocity vectors.

Other embodiments can involve a method of conducting falls risk assessments in which a plurality of adaptive thresholds are calculated based on angular velocity data from a plurality of shank-mounted inertial (also referred to as kinematic sensors). Inertial sensors are a subset of kinematic sensors, and the method may work for all forms of kinematic sensors where applicable. A plurality of initial contact points (such as heel strike points) and terminal contact points (such as toe-off points) may be detected based on the angular velocity data.

The TUG time segment that may be based on the angular velocity data includes at least one of a walk time, a turn time, and a return time. The walk time can identify an amount of time between a first step and a last step of a TUG test; the turn time can identify an amount of time between the first step and a turn step of the TUG test; and the return time can identify an amount of time between the turn step and the last step of the TUG test. Calculation of the TUG time segment is discussed in more detail below.

In addition, the method may involve calculating a derived parameter based on the angular velocity data. The derived parameter includes at least one of a temporal gait parameter, a tri-axial angular velocity-based parameter, a spatial gait parameter, and a turn parameter. The four categories of parameters are discussed below in more detail.

Additional parameters may also be obtained. For example, a test participant's stride length, stride velocity, and the coefficient of variation of both parameters may be derived based on, for example, gyroscopes attached to each of the participant's legs. In another example, parameters that measure the participant's ability to turn may also be derived. These parameters are discussed below in more detail. In another example, a participant's grip strength may be measured, such as with a handheld dynamometer. A participant's eyesight may also be measured, such as on a Binocular logmar or a Pelli-Robson contrast sensitivity scale. The participant's age and weight may also be recorded.

FIG. 1A shows an individual 10 performing a timed up and go (TUG) test in which the individual 10 gets up from a chair, walks three meters, turns at a designated spot 12, returns to the chair, and sits down. TUG time segments may be collected from test participants such as individuals from hospital in-patients, from nursing home residents, from community dwelling elderly adults, or from the general population. The test participant may also be evaluated using the Berg Balance Scale (BBS) to provide another measure of falls risk. Data relating to falls risk may be acquired by video, sensors, or both. In the illustrated example, a pair of wireless inertial sensors 14 (14a-14b) are coupled to the shanks (e.g., shins) 16 (16a-16b) of the legs of the individual 10, and output angular velocity data that can be used to automatically generate falls risk assessments. Thus, the illustrated approach could be used in primary or community care settings and may generate parameters that can be used to predict patients' falls risk. For example, some inertial sensor-based parameters described herein may enable automated measurement vestibular impairment, muscular strength, etc., and might be used to identify deficits in one or more of these areas.

Each sensor 14, which might be mounted to the corresponding shank 16 below the patella via a tight fitting piece of clothing, a sock, an elastic tubular bandage, embedded in a shoe, or any other method of attachment that can yield a clear tri-axial angular velocity signal, may include a tri-axial accelerometer and an add-on tri-axial gyroscope board. In particular, each sensor 14 may be positioned such that its measuring axis is aligned with the medio-lateral axis of the corresponding shank 16, and so that it is about half-way along the imaginary line between the Tibial Tuberosity (TT) and the Lateral Malleoulus (LM). In order to ensure that the angular velocity signal derived from the gyroscope has the correct polarity, the “skewness” of the signal (e.g., a measure of the asymmetry of the signal) may be calculated for each walk. If the skewness is less than zero, the gyroscope signal can be inverted to ensure the correct polarity of the signal. The sensors 14 may be programmed to sample each axis at a particular rate (e.g., 102.4 Hz) using firmware (e.g., TinyOS) or other programmable technique, and to wirelessly transmit the angular velocity data using a protocol such as a low-rate wireless PAN (personal area network) or Bluetooth protocol. Data may be streamed to various platforms, such as a desktop computer, laptop, or mobile device (e.g., a cellular phone).

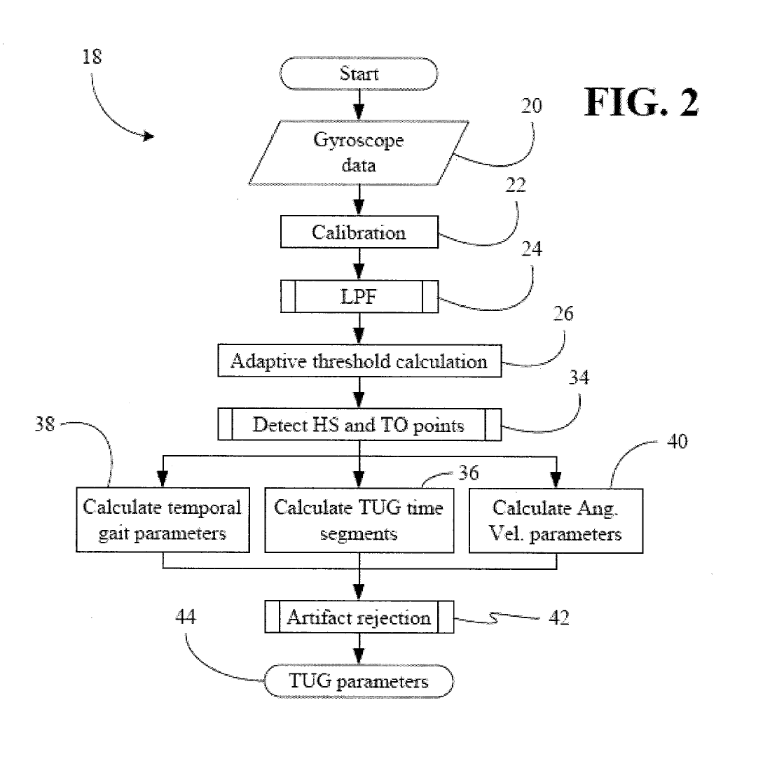

Turning now to FIG. 2, a method 18 of generating quantitative TUG parameters from gyroscope data is shown. The method 18 may be implemented in executable software as a set of logic instructions stored in a machine- or computer-readable medium of a memory such as random access memory (RAM), read only memory (ROM), programmable ROM (PROM), firmware, flash memory, etc., in fixed-functionality hardware using circuit technology such as application specific integrated circuit (ASIC), complementary metal oxide semiconductor (CMOS) or transistor-transistor logic (TTL) technology, or any combination thereof. For example, computer program code to carry out operations shown in method 18 may be written in any combination of one or more programming languages, including an object oriented programming language such as Java, Smalltalk, C++ or the like and conventional procedural programming languages, such as the “C” programming language or similar programming languages.

In the illustrated example, gyroscope data can be obtained using any appropriate mode of kinematic data acquisition. Upon receipt of the gyroscope data 20, processing block 22 may provide for using a sensor-to-segment offset orientation matrix (e.g., a rotation matrix) to calibrate the data 20 to derive acceleration and angular velocity vectors with respect to the coordinate axis of each inertial sensor. Illustrated block 24 applies a low pass filter (LPF) to the calibrated data. In one example, the LPF might include a zero-phase 5^{th} order Butterworth filter (e.g., 20 Hz corner frequency or 50.2 Hz corner frequency).

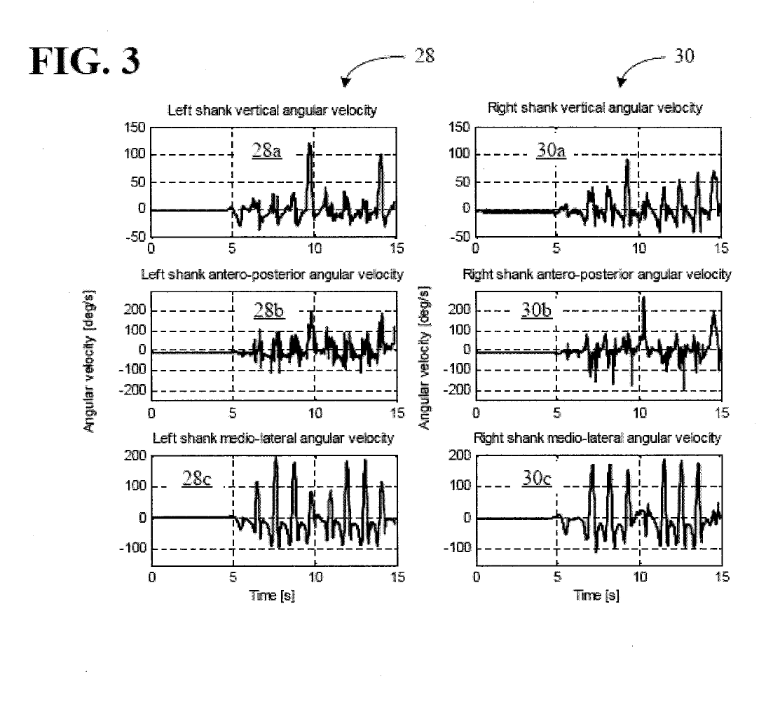

With reference to FIG. 3, a set of left shank signal plots 28 (28a-28c) and a set of right shank signal plots 30 (30a-30c) are shown. The plots 28 and 30 may represent tri-axial angular velocities corresponding to the motion of the individual 10 (FIG. 1) during the TUG test. In particular, plots 28a and 30a can be associated with Z-sensor axis angular velocity, plots 28b and 30b can be associated with X-sensor axis angular velocity, and plots 28c and 30c can be associated with medio-lateral (ML, Y-gyroscope sensor axis) angular velocity. Generally, the data corresponding to the plots 28 and 30 may be used to detect events such as initial contact points and terminal contact points, which may in turn be used to calculate quantitative TUG time segments and various related temporal gait parameters, as will be discussed in greater detail. The data corresponding to the plots 28 and 30 can also be used to calculate and/or derive other angular velocity-based parameters useful in the falls risk assessment analysis.

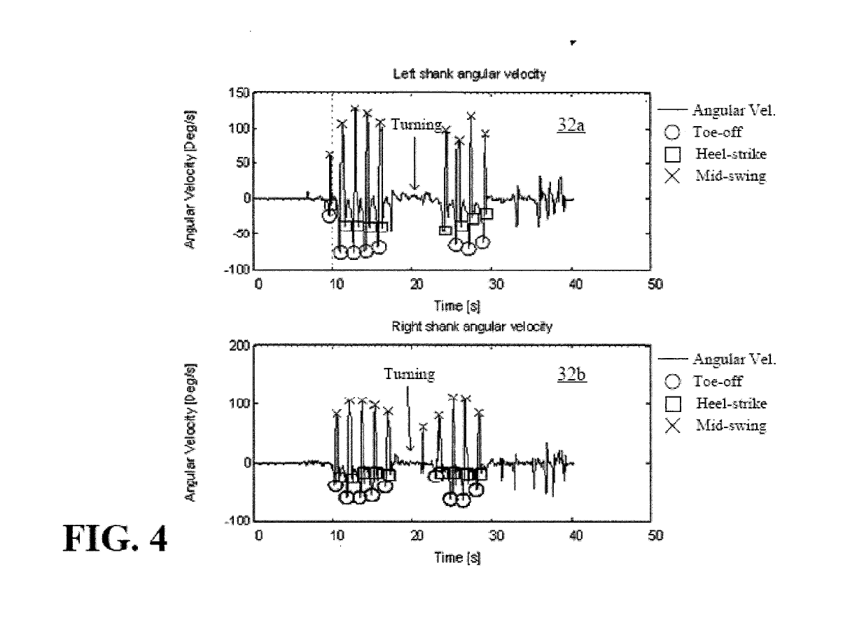

For example, FIG. 4 shows a pair of medio-lateral angular velocity plots 32 (32a-32b) in which a series of initial contact points and terminal contact points can be detected from the signals. Generally, each terminal contact point is reflected in a minimum value in the corresponding signal and is followed by a mid-swing point that can be identified via a maximum value in the signal. Each mid-swing point may then be followed by an initial contact point that is reflected in another minimum value. In addition, turning points may be detected from a period of minimum amplitude in the signal between periods of cyclical activity.

Returning to FIG. 2, block 26 demonstrates that a plurality of adaptive thresholds may be created based on the angular velocity data, wherein the adaptive thresholds can be used to define the likely range of the initial contact and terminal contact points in the medio-lateral angular velocity data. Thus, restricting the angular velocity data based on the adaptive thresholds can ensure robust detection of these points over a variety of walking speeds. In particular, the following adaptive thresholds might be used:

Mid-swing point for each gait cycle: valid local maximum peaks may be required to have a preceding minimum of at least th₁ rad/sec less than the maximum medio lateral angular velocity (ω_{ML}), wherein th₁ can be calculated as,

th₁=0.6·max(ω_{ML}) (1)

In addition, valid local maximum peaks can be required to be greater than th₂ rad/sec, wherein th₂ may be calculated as,

$${th}_{2} = 0.8 \cdot \frac{1}{N} {\sum }_{i = 1}^{N} ( {ω}_{{ML}_{i}} > {ϖ}_{ML} ) \qquad ( 2 )$$

Moreover, if two maximum peaks are found within t₁ seconds of each other, only the greater maximum can be considered, wherein t₁ may be defined as, for example, 0.5 seconds or fₛ*1.5 and fₛ is defined as the stride frequency.

Initial contact points: valid local minimums may be required to have a preceding maximum of at least th₃ rad/sec greater than the local minimum, wherein th₃ can be calculated as,

$${th}_{3} = 0.8 \cdot \vert \frac{1}{N} {\sum }_{i = 1}^{N} ( {ω}_{{ML}_{i}} < {ϖ}_{ML} ) \vert \qquad ( 3 )$$

In addition, valid local minimums could be required to be less than th₅, wherein th₅ might be defined as,

th₅=mean(ω_{ML}) (4)

Terminal contact points: valid local minimums can be required to be less than th₄, wherein th₄ may be calculated as,

$${th}_{4} = 0.8 \cdot \frac{1}{N} {\sum }_{i = 1}^{N} ( {ω}_{{ML}_{i}} < {ϖ}_{ML} ) \qquad ( 5 )$$

In addition, valid local minimums could be required to have a preceding maximum of at least th₆ greater than the local minimum, wherein th₆ might be defined as,

th₆=2th₃ (6)

Initial contacts and terminal contacts: following mid-point detection, only data within t₂ seconds may be considered, wherein t₂ can be defined as, for example, 1.5 seconds or fₛ*1.5. Specific values and ranges are provided herein to facilitate discussion only, and other values and ranges may be used as appropriate.

Block 34 may provide for detecting initial contact and terminal contact points based on the adaptive thresholds, as already discussed.

One or more quantitative TUG time segments can be calculated at block 36. The quantitative TUG time segments could include the walk time, the turn time and/or the return time. The walk time may identify the amount of time between the first step and the last step of the TUG test. The first step can be defined by at least one of the first initial contact point and the first terminal contact point, and the last step can be defined by at least one of the last initial contact point and the last terminal contact point. The turn time can identify the amount of time between the first step and the turn step of the TUG test. The turn step may be defined by at least one of a turn initial contact point and a turn terminal contact point. A lower ML angular velocity signal or a large positive peak in the vertical angular velocity may indicate that a TUG participant was turning at that point. In one example, a per-shank turn time is calculated for each shank as the time of the median detected gait point (terminal contact, heel-strike, mid-swing), and an overall turn time is calculated as the mean of the per-shank turn times. The return time may identify the amount of time between the turn step and the last step of the TUG test. Thus, the walk, turn and return times can be considered as time “segments” in that each calculation is a portion of the traditional quantitative TUG time, which is the entire amount of time required for the individual to complete the TUG test. The walk time, turn time and return time can be strong indicators of falls risk.

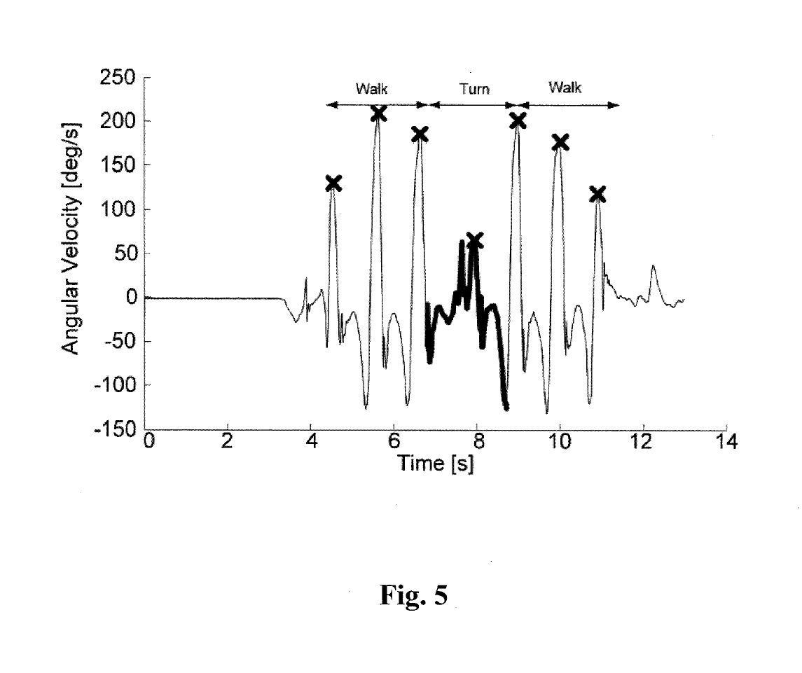

The turn time may also be used to examine the turning phase of the TUG test. The ML angular velocity signal may be automatically segmented into a walking section and a turning section. If the amplitude of a given mid-swing point was more than one standard deviation below the mean amplitude of all mid-swing points, it may be considered part of the turn. FIG. 5 illustrates this approach. The turning phase may be defined as the section of the signal that starts at the last initial contact point before the first mid-swing in the turn, and ending at the first terminal contact point after the last mid-swing in the turn. The turn may be quantified using the turn time and a number of steps taken for the subject to turn, as well as the ratio given by the number of steps taken to turn divided by the time taken to turn.

In addition to the TUG time segments, one or more derived parameters may also be determined. For example, block 38 demonstrates that the derived parameters could include various other temporal gait parameters. Examples of such temporal gait parameters include, but are not limited to, stride velocity, stride length, the number of gait cycles, the number of steps taken, cadence, step time, stride time, stance time, swing time, single support percentage, and double support percentage.

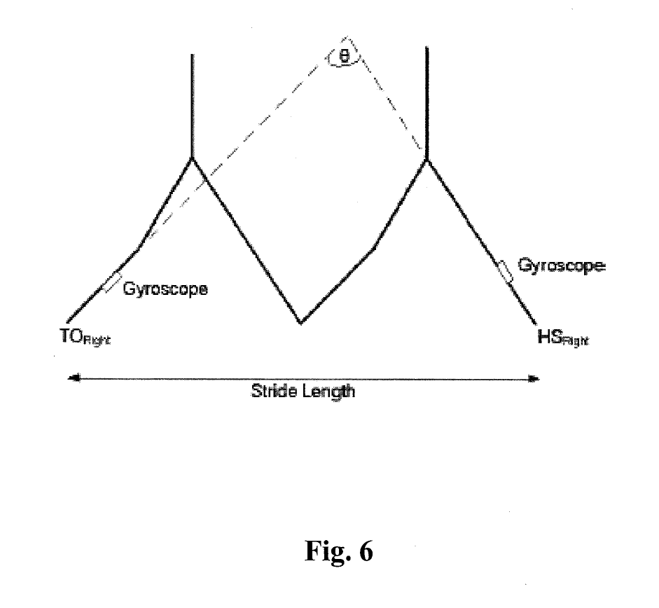

The stride length may be defined as the distance covered in a stride time. The stride time may include the time recorded between successive initial contact points (e.g., between successive heel strikes). The distance may be calculated as the distance covered during the swing phase of the gait cycle, which encompasses the time between a terminal contact point and a subsequent initial contact point. The stride length may be modeled as SxHx\sqrt{2(1−cos θ)}, where S represents a scaling factor to be optimized during calibration, H represents the participant's height, and θ represents the range of angular displacement in the sagittal plane during the stride. FIG. 6 illustrates a model of the stride length. The stride velocity may be calculated as stride length divided by stride time.

The number of gait cycles can be calculated as the number of initial contact points detected from the angular velocity signal during the TUG test minus one (i.e., the number of complete gait cycles).

The cadence (e.g., steps per minute) can be calculated as sixty times the number of steps taken while performing the TUG test divided by the walk time (e.g., time taken to take the steps identified during the TUG test).

$$Cadence = 60 \cdot ( \frac{\# Steps}{WalkTime} ) \qquad ( 7 )$$

Step time can be calculated as the time between the initial contact point on one foot and the initial contact point on the other foot. Stride time can be calculated from the time from initial contact (e.g., initial contact) of one foot to initial contact of the same foot.

Stance time can be calculated from the time between a initial contact and a terminal contact point on the same foot. Swing time can be calculated from the time between a terminal contact point and a initial contact point on the same foot. Double support may be determined by calculating the percentage of each gait cycle during which both feet are in contact with the ground (where the gait cycle time can be the time between successive right initial contacts). As will be discussed below, the number of gait cycles, number of steps taken, cadence, double support percentage and step time can all be strong indicators of falls risk either alone or in combination with one or more other effects.

Other temporal gait parameters that may be derived include single support variability, step time variability, swing time variability, walk-turn time ratio, TUG recording time, walk time, turn time, and return time.

Single support percentage for a foot may be defined as the swing duration of the other foot expressed as a percentage of gait cycle time, where the single support percentage data for each foot may be merged. The coefficient of variability (CV) for the single support percentage (as well as the other temporal gait parameters) can be calculated as a measure of single support variability. Thus, a “CV single support” parameter (expressed as a percentage) could be defined as the ratio of the standard deviation to the mean of the single support percentage. Similarly, a “CV step time” parameter may be calculated to reflect the step time variability as the ratio of the standard deviation to the mean of the step time.

The swing time can be calculated as the time between a terminal contact (TC) point and the initial contact (IC) point on the same foot. Thus, the swing time variability (“CV swing time”) could be expressed as the ratio of the standard deviation to the mean of the swing time. The walk-turn time ratio could be defined as the ratio of the time to turn to the time from turn (e.g., unity indicates the same time taken to walk to and from the turn). The single support variability, step time variability and walk-turn time ratio may be indicators of falls risk, particularly if combined with one or more other effects. The TUG recording time may be calculated from the duration of the edited data recording for each TUG test.

Block 40 demonstrates that in addition to the temporal gait parameters, the derived parameters may include one or more parameters that are obtained directly from the angular velocity signal in the Y, X, and Z directions in order to capture characteristics of the signal during the TUG test in three dimensions. FIG. 1B shows Cartesian as well as rotational axes that may be used by the sensor. These directions may correspond to, for example, the medio-lateral (ML), antero-posterior (AP) and vertical (V) directions. These angular-velocity-based parameters could include parameters to detect and analyze the speed and timing of the turn during the TUG test. For example, the mean, minimum and maximum angular velocities (averaged across both shanks) during the walk, expressed in degrees per second, may each be determined in the Y, X, and Z axes (which may correspond to, for example, the ML, AP and V directions). The measurements may form a set of nine (i.e., 3×3) tri-axial angular velocity parameters.

The tri-axial set of angular velocities may also be multiplied by the height of the individual performing the TUG test in order to obtain a variable approximately proportional to the linear velocity of the shank. This approximation can be based on the formula for linear velocity, which equals the radius times angular velocity, wherein the radius is the leg length and height is assumed to be approximately proportional to the leg length. Thus, the linear velocity may be specifically related to the shank/foot of the individual as opposed to merely the trunk of the individual.

A mid-swing point and mean amplitude may also be calculated. The mean amplitude of the mid-swing points can be calculated as the mean angular velocity at each of the mid-swing points, while the range of mid-swing points may be defined as the difference in amplitude (in deg/s) between the largest and smallest mid-swing points on the angular velocity signal obtained for each shank Thus, the range of mid-swing point amplitudes may capture variability in leg movement.

The walk angular velocity, linear velocity and mid-swing point amplitude parameters can be strong indicators of falls risk either alone or in combination with one or more other effects. In addition, other angular velocity-based parameters such as turn angular velocity may be calculated. The turn angular velocity can be defined as the mean amplitude (taken across both shanks) of the angular velocity signal at the turn point for each shank. The turn angular velocity may be an indicator of falls risk, particularly if combined with one or more other effects. The coefficient of variation (CV) may also be calculated for each angular velocity parameter in order to provide a measure of variation during the TUG test. The CV may be calculated as a ratio of the standard deviation of the parameter measurements to the mean of the parameter measurements, expressed as a percentage. A non-exhaustive list of parameters that may be collected and derived is summarized in FIG. 7.

As already discussed, the temporal gait parameters and TUG time segments may be calculated from the gait characteristic points such as initial contact and terminal contact points. An artefact rejection routine may be employed at block 42 to remove spurious temporal parameters that might have been calculated from erroneous gyroscope data. The artefact rejection routine can also be designed to account for missing and extra IC and TC points detected by the adaptive TUG algorithm. Artefact rejection may be based on two strands: examining temporal sequence information, and examining times between successive characteristic points (e.g., “gait cycle information”).

Temporal sequence information may be obtained based on the following: once all characteristic points are detected in processing block 34, each point may be assigned a numerical label of one to four—1-right heel-strike, 2-left terminal contact, 3-left heel-strike, 4-right terminal contact. A correct gait cycle (if starting on a right initial contact) would then follow the sequence 1, 2, 3, 4. By subtracting each label from the previous label, spurious samples (e.g., samples not producing a difference equal to either −3 or 1) may be deemed artefacts and rejected.

Gait cycle information may be obtained based on the following: the time between adjacent gait characteristic points may be calculated for each set of characteristic points (e.g., right IC, left TC, left IC, right TC). This calculation can be referred to as “gait cycle time”. If the difference between any successive characteristic point is greater than a particular time threshold (e.g., 2.5 seconds), the associated characteristic point could be identified as an artefact. Similarly, if the difference between any successive characteristic point is zero seconds, the associated point may be flagged as an artefact. Furthermore, any gait parameters with a negative or zero value may also be rejected. The result may be a set of TUG parameters 44 that are highly reliable and can be used to effectively generate falls risk assessments.

Any video data for each test participant's TUG test may also be visually inspected to ensure that only data from valid TUG tests are included in assessing falls risk.

The gait and balance of community dwelling elderly adults, for example, may be assessed using shank-mounted inertial sensors while each of the adults perform the TUG test. Individuals may also be evaluated using the Berg balance scale (BBS), and the above-described TUG time segments and other derived parameters may be collected or calculated based on the angular velocity data from the inertial sensors, as discussed above. Table I below shows example mean and ranges of some of the parameters collected from test participants. The participants in the example involved 349 participants, consisting of 103 male participants and 246 female participants. The data analyzed were acquired from 207 participants with a self-reported history of falling and 142 participants without a self-reported history of falling. 65 of the participants had two or more falls in the previous year. 119 participants had no history of falls. The mean age of the participants was 72.4±7.4 years of age, and the mean weight was 73.7±14.5 kg. The manual TUG data shows strong variation with age. While manual TUG time in the example increased with age for both genders, and was longer for fallers than non-fallers, the differences between fallers and non-fallers did not vary with age. There was statistically significant difference between fallers and non-fallers in maximum grip strength over the whole group as well as in the male and females under 75 groups. Contrast sensitivity was also significantly different between fallers and non-fallers overall and in the male group. Binocular logmar, on the other hand, was not found to be significant overall in any of the sub-groups.

TABLE I

| | Faller | Faller | Non-Faller | Non-Faller |
|---|---|---|---|---|
| Variable | Male (N = 44) | Female (N = 163) | Male (N = 59) | Female (N = 83) |
| Age (yrs) | 75.5 ± 7.53 | 73.63 ± 7.19 | 69.91 ± 6.67 | 69.84 ± 6.67 |
| Height (cm) | 172.51 ± 7.71 | 161.19 ± 7.71 | 175.19 ± 6.96 | 164.23 ± 7.24 |
| Weight (kg) | 77.95 ± 12.19 | 68.56 ± 13.22 | 83.51 ± 13.01 | 74.58 ± 14.67 |
| Binocular logmar | 1.54 ± 0.25 | 1.64 ± 0.17 | 1.70 ± 0.16 | 1.69 ± 0.14 |
| Contrast sensitivity | 0.18 ± 0.21 | 0.14 ± 0.17 | 0.12 ± 0.10 | 0.1 ± 0.11 |
| Max grip (lbs) | 64.45 ± 22.41 | 38.74 ± 12.79 | 79.93 ± 23.49 | 45.87 ± 15.97 |
| Manual TUG (s) | 11.37 ± 5.00 | 11.59 ± 5.25 | 8.11 ± 2.09 | 8.80 ± 2.94 |
| BBS | 49.71 ± 5.94 | 49.8 ± 7.33 | 54.23 ± 2.93 | 53.91 ± 3.28 |

Generally, the quantitative TUG time segment parameters may be strongly correlated with the manual TUG time, including return time (ρ=0.89, p<0.001), time of turn (ρ=0.83, p<0.001) and walk time (ρ=0.90, p<0.001). The parameters may have significance only in combination with another effect. Those parameters may therefore contain complementary information about the properties of standing, turning and walking associated with falls that are not captured by the BBS and manual TUG tests.

Generating classifier models based on the collected and calculated parameters may require the models to be trained based on the occurrence of falls experienced by the test participants. This may be done using a retrospective or a prospective approach.

In the retrospective approach, data on a participant's prior falls history may be collected, such as in the previous five years. A fall may be considered as a sudden, unintentional change in position causing an individual to land at a lower level, on an object, on the floor, on the ground, or other surface. A fall may be more generally considered as a loss in balance or a change in position that causes a person to drop toward the ground. In the retrospective approach, a faller may include participants who experienced a threshold number of falls, such as two or more falls in the past five years. A faller may additionally include participants who experienced certain risk factors during a fall, including syncope, presyncope or loss of consciousness; dizziness or light-headedness; chronic pain; injuries after falling; fear of falling; depression after falling, or some other characteristic related to falling. A participant who had an accidental fall without risk factors would be classified as a non-faller. For the classifier models, the participants may be classified, for example into those who are not at risk (no falls in last five years), those who are at risk (no falls but has problems with balance and walking), Faller 12-months (one fall in previous 12 months), Faller 6-months (one fall in previous six months) and Recurrent Faller (more than two falls in previous 12 months)

In the prospective approach, test participants may be contacted after their initial baseline assessment to collect falls data. For example, each participant may be contacted within two years of the initial baseline assessment to determine whether the participant had fallen during that time span. Participants with two or more falls in the follow-up period may be deemed recurrent fallers. The prospective approach may take more time to acquire data related to the occurrence of falls, but may be more clinically relevant and reliable than the retrospective approach because the self-reported falls collected during a retrospective approach may be unreliable.

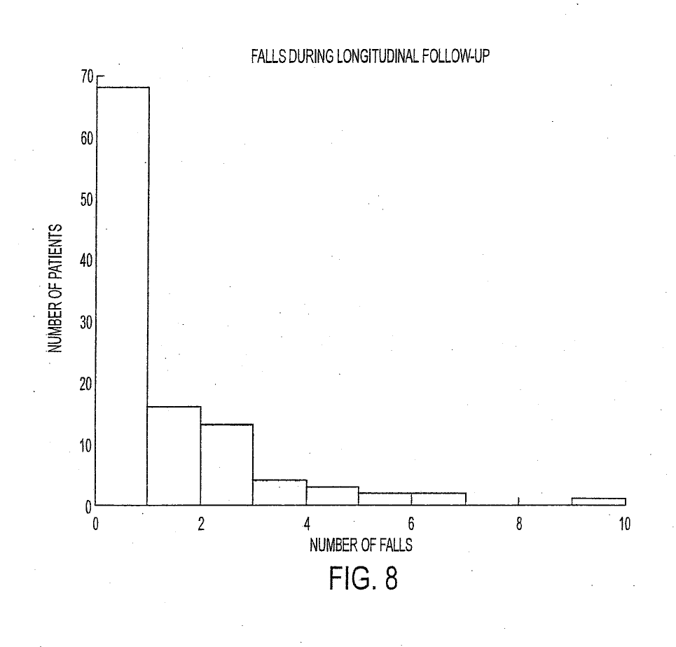

An initial study may generate TUG-derived parameters and use self-reported falls history to generate a retrospective falls risk estimate. A prospective approach may follow up with the test participants to determine which participants have experienced a fall after the initial study, and how many times they have fallen. In this prospective approach, the new data may be used to train a predictive classifier model to generate a falls risk estimate. FIG. 8 illustrates example falls data that was collected during a participant follow-up for the prospective approach. In the example, the 349 participants who were evaluated using the retrospective approach (referred to as baseline assessment) were contacted. Valid follow-ups were obtained for 299 participants (84 male participants, 215 female participants). In the example prospective approach, the participants experienced 189 falls since the TUG test. 109 participants experienced a fall, 39 participants had two or more falls, and 190 participants did not fall.

To identify those parameters of specific importance to an estimation of falls risk, an optional initial non-parametric screening may be performed. For example, the Mann-Whitney version of the Wilcoxon rank-sum test may be used to test for statistical differences between subjects who experienced a fall and those who did not. The Wilcoxon rank-sum test may test for statistical differences in each variable.

Following initial non-parametric screening, regularized discriminant (RD) statistical classifier models may be used to generate models for predicting risk of falling. Classifier models such as a linear discriminant classifier model, a quadratic discriminant classifier model, or a regularized discriminant classifier model may be used. A linear discriminant classifier model, for example, may be used if there is enough data to calculate a common covariance matrix. In general, the data set may be divided into classes. A class conditional mean vector uₖ may then be generated to calculate a covariance matrix and then a discriminant for the class. The class conditional mean vector uₖ may be calculated as

$${μ}_{k} = \frac{1}{( {N}_{k} )} {\sum }_{n = 1}^{{N}_{k}} {x}_{nk} ,$$

where xₙₖ is the nth feature vector in class k, N is the total number of feature vectors, and uₖ is the class conditional mean vector. The class conditional mean vector may be used to calculate a common covariance matrix Σ:

$$\sum = \frac{1}{N - c} {\sum }_{k = 1}^{c} {\sum }_{n = 1}^{{N}_{k}} ( {x}_{nk} - {μ}_{k} ) {( {x}_{nk} - {μ}_{k} )}^{T} ,$$

where c is the number of pattern classes.

The common covariance matrix may be used to calculate the discriminant yₖ(x):

yₖ(x)=−½μₖ^{T}Σ⁻¹μₖ+μₖ^{T}Σ⁻¹x+log(πₖ), where x is the feature vector and πₖ is the prior probability.

A quadratic discriminant classifier model may also be used. The covariance matrix for each class Σₖ of the model may be calculated as

$${\sum }_{k} = \frac{1}{{N}_{k} - 1} {\sum }_{n = 1}^{{N}_{k}} ( {x}_{nk} - {μ}_{k} ) {( {x}_{nk} - {μ}_{k} )}^{T} .$$

The covariance matrix may be used to calculate a quadratic discriminant for each class:

yₖ(x)=−(x−μₖ)^{T}Σ⁻¹(x−μₖ)+2 log(πₖ)−log|Σₖ⁻¹|

The class with the largest discriminant for all classifier models may be used as the final class.

In pattern recognition problems with small data sizes (i.e., small number of test participants) and a large feature set, however, some of the parameters are not always identifiable from the data because the covariance matrix can be singular (with zero or infinitesimal eigenvalues). Because such matrices are non-invertible, a linear discriminant model, as described above, may not be obtainable. Such problem is said to be ill posed. Regularization may be used as a solution by biasing the data set away from their sample values toward more physically plausible values. Methods for performing the regularization may be found in, for example, Combining pattern classifiers: methods and algorithms, by Kuncheva. There are two methods, which may be combined, for stabilizing the covariance matrix. In a first method, regularization may be performed towards common covariance matrix with parameters λ:

Σₖ(λ)=(1−λ)Σₖ+λΣ, where Σₖ is an estimate of the covariance matrix for a class k and E is the common covariance matrix.

In a second method, regularization may further be performed towards the diagonal matrix (with eigenvalues equal to the mean of the eigenvalues of the sample based estimate of the covariance matrix), with parameter r:

$${\sum }_{k} ( r ) = ( 1 - r ) {\sum }_{k} + \frac{r}{{n}_{c}} tr ( {\sum }_{k} ) I ,$$

where I is the n_{c}×n_{c} identity matrix and n_{c} is the dimension of the covariance matrix Σₖ.

Combining the two methods yields a combined regularization formula:

$${\sum }_{k} ( λ , r ) = ( 1 - r ) {\sum }_{k} ( λ ) + \frac{r}{{n}_{c}} tr ( {\sum }_{k} ( λ ) ) I$$

The discriminant function used in regularized discriminant analysis may be calculated using the new estimate for the class conditional covariance matrices and the quadratic discriminant formula. Regularization parameters of λ=1 and r=0 correspond to a linear discriminant classifier model while λ=0 and r=0 correspond to a quadratic discriminant classifier model. The optimum classifier model may be determined by finding the regularization parameters that yielded the largest value of classification accuracy (lowest value of mean classification error obtained through cross-validation).

To account for differing class proportions, the weighting of the training data for each participant by the (faller and non-faller) class proportions may be implemented. This may be accomplished by setting the prior probability for a given class k equal to the proportion of that recording labeled as class k. Prior to training, features may be normalized to have zero mean and unity standard deviation. These normalizing data may then be applied to, the testing data. Each case in the testing set may then be classified by assigning it to the class with the largest value of the discriminant function.

Feature selection may subsequently be performed, such as through a filter method or a wrapper method. Filter methods rely on general characteristics of the data, such as correlation with class labels, to evaluate and select the feature subsets without involving the classifier algorithm. Wrapper methods use the performance of the classifier on the given dataset to evaluate each candidate feature subset. Wrapper methods may search for a more optimal feature set for a given classifier algorithm. Unlike filter based methods, a wrapper-based method may consider interactions between features and may contain less redundancy. The filter method may use a nearest neighbor criterion to add and remove features from the feature subset. A wrapper method, such as sequential forward feature selection, may sequentially add features to an empty set until the addition of further features does not increase the classification accuracy.

Applying this technique not only produces parameters for a probability estimate of the risk of future falls, but also isolates parameters which may be related to specific function deficits, such as physical and sensory deficits. For example, selection of minimum ground clearance (MGC), or angular velocity parameters related to MGC (e.g., selection of mean absolute-valued vertical angular velocity and acceleration), in the feature set could indicate that the test participant associated with the model had poor core or lower limb strength, which is often related to low MGC values. For example, selection of the MGC or related angular velocity parameters informs a clinician of what aspects of a participant's movement places that person at risk of falls. The diagnosis may be tailored to an individual participant, and allows for tailored intervention or treatment to prevent future falls.

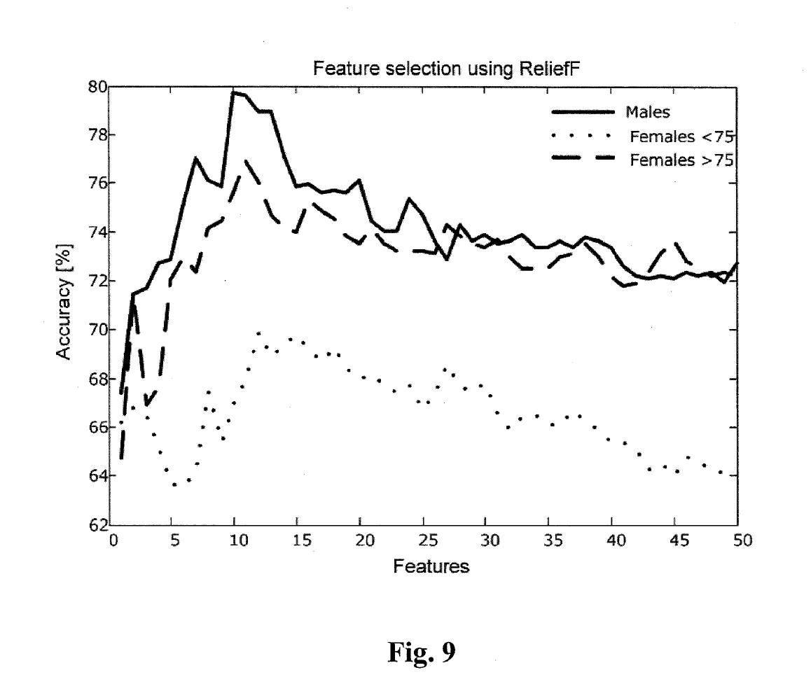

An optimum classifier model may be developed for subsets of the data. For example, a first classifier model may be developed for all male test participants. A second classifier model may be developed for all female test participants under the age of 75, and a third classifier model may be developed for all female test participants over the age of 75. FIG. 9 illustrates example results of a feature selection performed, using a filtering feature selection routine and a retrospective approach. The figure shows the variation of the optimum classification accuracy with feature number (where feature number is the number of features included in the model). Optimum accuracy using the feature selection for the male model was obtained with 15 features. Optimum accuracy using the feature selection for females under 75 model was obtained with 19 features, and optimum accuracy for the females over 75 model was obtained with four features.

After the data is classified by gender and age, a grid search may be carried out to determine optimum feature set (using each feature selection method) and model parameters (λ and r) for each of the models. For example, when the data set is stratified (e.g., into data taken from male participants, data from female participants under 75 years of age, and data from female participants over 75 years of age) the optimum feature set and model parameters may be determined for each of the three models. This operation may attempt to determine the optimum classifier configuration in terms of features and classifier model parameters employed in each of the models for both methods of feature selection.

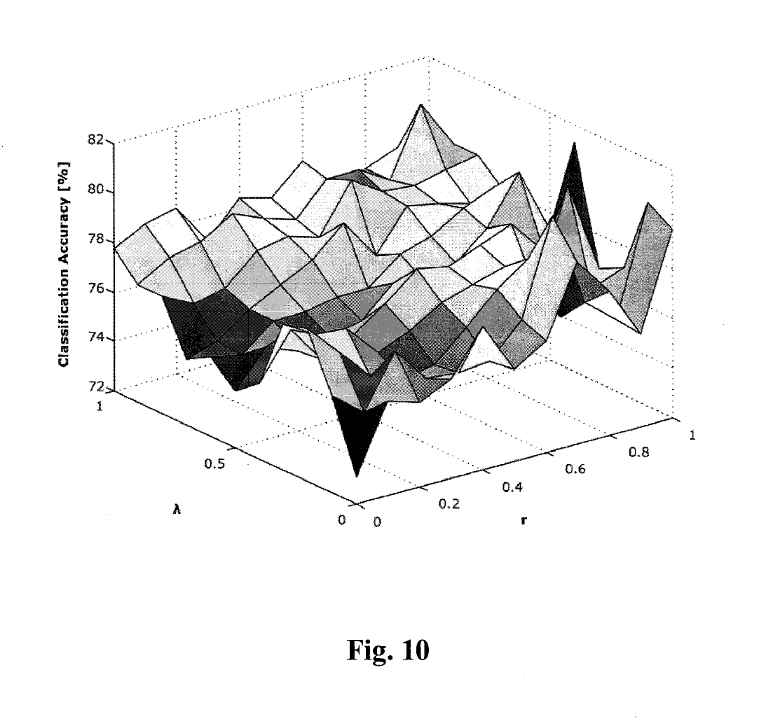

FIG. 10 shows example results of a grid search to determine the optimum classifier parameters (i.e., λ and r) for each feature set obtained through sequential forward feature selection. The results are based on a retrospective approach and shown graphically. The regularization parameters of λ=0.4 and r=1 are shown to yield optimum performance for the particular grid search for a classifier model for male test participants.

The process above generates optimum features and classifier model parameters. Table IIA lists example optimum parameters that were determined using a retrospective approach. Table IIB lists example optimum parameters that were determined using a prospective approach.

TABLE IIA

| Model 1 (Male) | Model 2 (Female <75) | Model 3 (Female ≧75) |
|---|---|---|
| CV Double support | Mean single support | Mean single support |
| (%) | (%) | (%) |
| CV step time (%) | Mean stance time (s) | Mean stance time (s) |
| No. Steps | CV step time (%) | Mean mid-swing points |
| | | (deg/s) |
| CV X-axis Angular | Age (yrs) | CV X-axis angular |
| velocity (%) | | velocity (%) |
| Min X-axis angular | Weight (kg) | Mean Y-axis angular |
| velocity × Height | | velocity × Height |
| BMI | Min Y-axis ang. Vel. | Max Y-axis angular |
| | (deg/s) | velocity × Height |
| Contrast Sensitivity | Max Y-axis ang. Vel. | Min Z-axis angular |
| | (deg/s) | velocity × Height |
| | Min X-axis ang. Vel. × | Age (yrs) |
| | Height | |
| | Max Grip (N) | Contrast Sensitivity |

TABLE IIB

| Model 1 (Male) | Model 2 (Female <75) | Model 3 (Female ≧75) |
|---|---|---|
| Mean stance time | Mean swing time (s) | CV Double support (%) |
| CV step time (%) | Turn ang. vel. (deg/sec) | CV single support (%) |
| Range of mid-swing | Min Z-axis Ang. Vel. | Mean stance time (s) |
| points (deg/s) | (deg/sec) | |
| Return time (s) | CV velocity (%) | CV swing time (%) |
| CV Z-axis Ang. | Mean Turning Time (s) | Cadence (steps/min) |
| Vel. (%) | | |
| Weight | Min X-axis ang. Vel. × | CV Z-axis Ang. Vel. (%) |
| | Height | |
| | Max Z-axis ang. Vel. × | Mean Turning Time (s) |
| | Height | |
| | Min Z-axis ang. Vel. × | Max Z-axis ang. |
| | Height | Vel. × Height |
| | | Age (yrs) |
| | | Weight (kg) |
| | | Height (cm) |
| | | BMI |

The performance of the algorithm may be estimated using cross validation. For example, the data may be randomly split into 10 equal sections or folds. Nine of the folds may be used to train the classifier and the remaining fold may then be used to test the performance of the classifier. Repeating this procedure 10 times and taking the mean results in an unbiased, low variance estimate of the classifier's performance.

Metrics for the accuracy of the classifier includes the accuracy (Acc), Sensitivity (Sens) and specificity (Spec). Sensitivity can be defined as the proportion of fallers (as labeled by the geriatrician evaluating the subject in the clinic) correctly identified by the model. Similarly, specificity can be defined as the proportion of non-fallers that are correctly identified by the model. Accuracy can then be defined as the overall percentage of patients correctly classified. Receiver operating characteristic (ROC) curves may be generated for each classifier model using the test set probability outputs obtained by cross validation. The area under the ROC curve may be used as an index of each statistical model's performance. Table IIIA below shows example accuracy data for classifier models trained using a retrospective approach. There, the method on average correctly classified 81.32% of participants with and without a history of falls. Table IIIB shows example accuracy data for classifier models trained using a prospective approach. There, the method on average correctly classified 79.05% of participants with and without a history of falls.

TABLE IIIA

| | Model 1 | Model 2 | Model 3 | Mean | BBS | mTUG |
|---|---|---|---|---|---|---|
| Acc | 81.17 | 78.09 | 84.71 | 81.32 | 60.74 | 60.55 |
| (%) | | | | | | |
| Sens | 72.19 | 87.14 | 88.94 | 82.76 | 44.53 | 46.80 |
| (%) | | | | | | |
| Spec | 87.56 | 64.00 | 76.09 | 75.88 | 84.17 | 80.49 |
| (%) | | | | | | |
| ROC | 0.76 | 0.78 | 0.83 | 0.79 | 0.71 | 0.73 |
| area | | | | | | |

TABLE IIIB

| | Model 1 | Model 2 | Model 3 | Mean | BBS | mTUG |
|---|---|---|---|---|---|---|
| Acc | 84.52 | 73.18 | 79.45 | 79.05 | 66.01 | 60.27 |
| (%) | | | | | | |
| Sens | 83.18 | 65.24 | 78.95 | 75.79 | 42.80 | 39.27 |
| (%) | | | | | | |
| Spec | 85.25 | 78.09 | 79.72 | 81.02 | 79.35 | 72.38 |
| (%) | | | | | | |
| ROC | 0.86 | 0.72 | 0.75 | 0.77 | 0.63 | 0.62 |
| area | | | | | | |

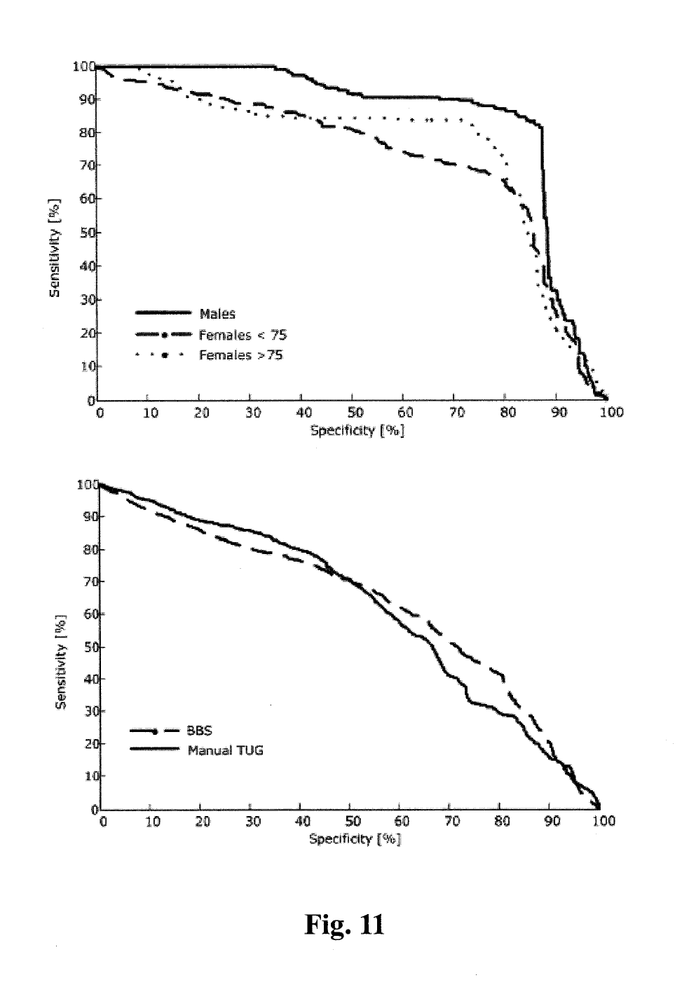

The top panel in FIG. 11 shows example ROC curves for each of the three gyroscopes derived classifier models using a prospective approach while the bottom panel shows the ROC curves constructed for the models derived from the gyroscopes parameters compared to those derived solely from the manual TUG time and BBS score. The curves provide a graphical illustration of the performance of the system when compared to standard falls risk assessments.

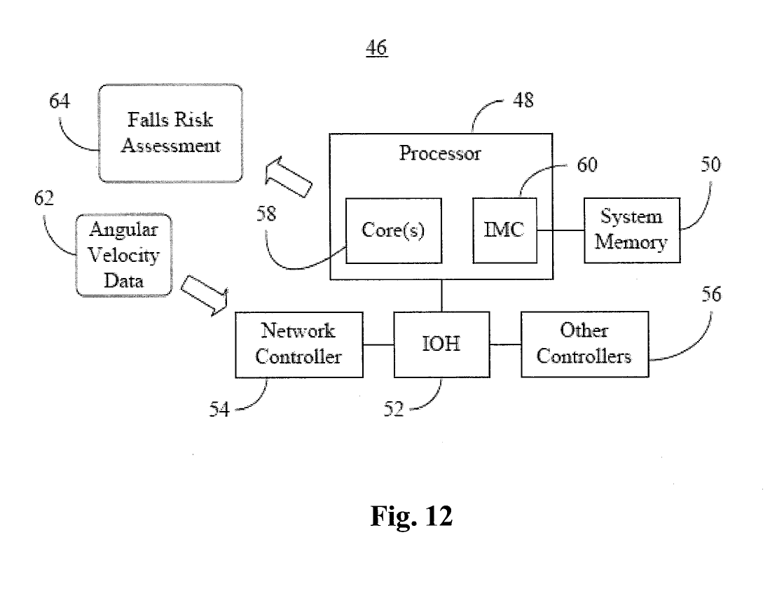

Turning now to FIG. 12, a computing system 46 is shown having a processor 48, system memory 50, an input/output hub (IOH) 52, a network controller 54, and various other controllers 56. The system 46 could be part of a mobile platform such as a laptop, personal digital assistant (PDA), mobile Internet device (MID), wireless smart phone, media player, imaging device, etc., or any combination thereof. For example, the system 46 might be implemented in a wireless smart phone carried by an individual performing a TUG test in a primary care, community care or home setting. In addition, the system 46 may also be part of a fixed platform such as a personal computer (PC), server, workstation, etc. Thus, the processor 48 may include one or more processor cores 58 capable of executing a set of stored instructions, and an integrated memory controller (IMC) 60 configured to communicate with the system memory 50. The system memory 50 could include dynamic random access memory (DRAM) configured as a memory module such as a dual inline memory module (DIMM), a small outline DIMM (SODIMM), etc.

The illustrated IOH 52, sometimes referred to as a Southbridge of a chipset, functions as a host device and communicates with the network controller 54, which could provide off-platform communication functionality for a wide variety of purposes such as cellular telephone (e.g., W-CDMA (UMTS), CDMA2000 (IS-856/IS-2000), etc.), WiFi (e.g., IEEE 802.11, 1999 Edition, LAN/MAN Wireless LANS), Low-Rate Wireless PAN (e.g., IEEE 802.15.4-2006, LR-WPAN), Bluetooth (e.g., IEEE 802.15.1-2005, Wireless Personal Area Networks), WiMax (e.g., IEEE 802.16-2004, LAN/MAN Broadband Wireless LANS), Global Positioning System (GPS), spread spectrum (e.g., 900 MHz), and other radio frequency (RF) telephony purposes. In the illustrated example, the network controller 54 obtains angular velocity data 62 wirelessly (e.g., from a data aggregator over a Bluetooth connection), and provides the angular velocity data 62 to the processor 48 for further analysis. The illustrated processor 48 calculates TUG parameters 44 (FIG. 2) and generates falls risk assessments 64, which might also be gender, weight and/or age-based, as already discussed.

The other controllers 56 could communicate with the IOH 52 to provide support for user interface devices such as a display, keypad, mouse, etc. in order to allow a user to interact with and perceive information from the system 46.

Embodiments of the present invention are applicable for use with all types of semiconductor integrated circuit (“IC”) chips. Examples of these IC chips include but are not limited to processors, controllers, chipset components, programmable logic arrays (PLA), memory chips, network chips, and the like. In addition, in some of the drawings, signal conductor lines are represented with lines. Some may be thicker, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments of the present invention are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments of the invention. Further, arrangements may be shown in block diagram form in order to avoid obscuring embodiments of the invention, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments of the invention, it should be apparent to one skilled in the art that embodiments of the invention can be practiced without, or with variation of, these specific details. The description is thus to be regarded as illustrative instead of limiting.

The term “coupled” is used herein to refer to any type of relationship, direct or indirect, between the components in question, and may apply to electrical, mechanical, fluid, optical, electromagnetic, electromechanical or other connections. In addition, the terms “first”, “second”, etc. are used herein only to facilitate discussion, and carry no particular temporal or chronological significance unless otherwise indicated.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

We claim:

1. A falls risk assessment method comprising:
calculating a plurality of kinematic parameters based on angular velocity data from a plurality of shank-mounted kinematic sensors obtained during a timed up and go (TUG) test;
generating a regularized discriminant classifier model based on at least one of the kinematic parameters;
performing sequential forward feature selection to base the regularized discriminant classifier model on an additional parameter of the plurality of kinematic parameters; and
performing a grid search to generate at least one optimum parameter for the regularized discriminant classifier model.

2. The method of claim 1, further comprising receiving falls data at a time after the TUG test; and adjusting the regularized discriminant classifier model based on the received falls data.

3. The method of claim 2, further comprising identifying, from the parameters selected for the regularized discriminant classifier model, one or more physical or sensory deficits related to walking.

4. The method of claim 1, wherein the TUG parameters comprise at least a cadence, a number of gait cycles, a number of steps taken, a set of tri-axial angular velocities, a set of tri-axial linear velocities, and a mid-swing point angular velocity.

5. The method of claim 3, further comprising multiplying the set of tri-axial angular velocities by a height of an individual to obtain a set of tri-axial linear velocities.

6. The method of claim 1, further comprising determining an accuracy of the regularized discriminant classifier model based on performing class validation.

7. A system comprising:
a plurality of kinematic sensors to be coupled to a corresponding plurality of shanks of an individual;
a processor; and
a memory to store a set of instructions which, if executed by the processor, cause the system to,
calculate a timed up and go (TUG) time segment based on angular velocity data from the plurality of kinematic sensors;
calculate a plurality of derived parameters based on the angular velocity data;
generate a regularized discriminant classifier model based on the TUG time segment, based on one of the plurality of derived parameters, or based on any combination thereof;
perform a sequential forward feature selection to base the regularized discriminant classifier model on an additional parameter of the plurality of derived parameters; and
perform a grid search to generate at least one optimum parameter for the regularized discriminant classifier model.

8. The system of claim 7, wherein the instructions which, if executed by the processor, further cause the system to
receive falls data at a time after the TUG test; and adjust the regularized discriminant classifier model based on the received falls data.

9. The system of claim 7, wherein the TUG parameters comprise at least a cadence, a number of gait cycles, a number of steps taken, a set of tri-axial angular velocities, a set of tri-axial linear velocities, and a mid-swing point angular velocity.

10. The system of claim 7, wherein the instructions, if executed, further cause the system to:
multiply the set of tri-axial angular velocities by a height of an individual to obtain a set of tri-axial linear velocities.

11. The system of claim 7, wherein the instructions, if executed, further cause the system to:
determine an accuracy of the regularized discriminant classifier model based on performing class validation.

12. A computer readable storage medium comprising a set of instructions which, if executed by a processor, cause a computer to:
calculate a timed up and go (TUG) time segment based on angular velocity data from the plurality of kinematic sensors;
calculate a plurality of derived parameters based on the angular velocity data;
generate a regularized discriminant classifier model based on the TUG time segment, based on one of the plurality of derived parameters, or based on any combination thereof;
perform a sequential forward feature selection to base the regularized discriminant classifier model on an additional parameter of the plurality of derived parameters; and
perform a grid search to generate at least one optimum parameter for the regularized discriminant classifier model.
